(19) United States
(12) Patent Application Publication
(10) Pub. No.: US 2010/0160854 A1
(43) Pub. Date: Jun. 24, 2010

(54) INTEGRATED PATIENT MANAGEMENT AND CONTROL SYSTEM FOR MEDICATION DELIVERY

(76) Inventor: Robert T. Gauthier, San Diego, CA (US)

Correspondence Address: O''Melveny & Myers LLP;IP&T Calendar Department LA-13-A7, 400 South Hope Street, Los Angeles, CA, 90071-2899, US

(21) Appl. No.: 12/643,398

(22) Filed: Dec. 21, 2009

Related U.S. Application Data
(60) Provisional application No. 61/139,826, filed on Dec. 22, 2008.

(51) Int. Cl. A61M 5/142; A61B 5/153; A61B 17/132

(52) U.S. Cl. 604/31; 600/584; 606/203

(57) ABSTRACT

An integrated patient monitoring and control system is provided which includes a SITS, the SITS being adapted for coupling to the patient to obtain a specimen from the patient, a sensor, the sensor being adapted to receive the specimen from the SITS and to analyze the sample, a medication control unit, the medication control unit receiving information from the sensor, and utilizing that information to determine medication dosing information for the patient, and a medication administration system, the medication administration system receiving the dosing information from the medication control unit, and adapted to cause administration of the medication to the patient. If the SITS is adapted for blood draw, the system advantageously is performed in conjunction with a pneumatic pressure cuff, inflated so as to aid in blood draw.

### RELATED APPLICATION INFORMATION

This application claims priority to and benefit of U.S. Provisional Application Ser. 61/139,826, filed Dec. 22, 2008, entitled “Automated Blood Sampling Systems And Methods”, the content of which is incorporated by reference herein in its entirety as if fully set forth herein.

This application is related to U.S. Provisional Application Ser. No. 61/086,383, filed Aug. 5, 2008 (our Reference 037,028-002); U.S. Utility application Ser. No. 12/534,447, filed Aug. 3, 2009 (our Reference 037,028-006); U.S. Provisional Application Ser. No. 61/171,904, filed Apr. 23, 2009 (Our Reference 037,028-004); and U.S. Provisional Application Ser. No. 61/172,433, filed Apr. 24, 2009 (Our Reference 037,028-005), each of which are incorporated herein by reference in their entirety as if fully set forth herein.

### FIELD OF THE INVENTION

The invention relates generally to an automated closed loop (feedback controlled) drug delivery system using an optimal sampling method and control system. More particularly, the invention relates to methods and apparatus for use in the administration of drugs, such as heparin as an anti-coagulant medicine used in the treatment of cardiovascular and neurovascular disease as well as deep-vein thrombosis and pulmonary embolic disease.

### BACKGROUND OF THE INVENTION

Millions of patients are treated with unfractionated heparin (UFH) in the acute care hospital setting to control their level of anticoagulation. These patients are monitored by a multi-step, labor intensive process to maintain their level of anticoagulation. This complex process leads to frequent human error, thus only 35%-50% of patients are within a safe range of heparin at any given time. The consequences of both under- and over-anticoagulation include death, heart attack, stroke, moderate to severe blood loss, tremendous strain on the patient and their loved ones, and millions of dollars in avoidable health care costs. The problem has become so serious that the Joint Commission recently issued a “Sentinel Event Alert”¹ regarding the prevention of errors related to heparin. Such alerts require immediate investigation and response for an event that carries a significant chance of a serious adverse outcome. Several approaches have been tried to improve control of heparin levels. These approaches include point-of-care monitoring and use of standardized nomograms. The attempts have yielded little if any improvement.

Heparin, alone or in conjunction with other antithrombotic agents, is the standard of treatment in patients with acute myocardial infarction (AMI), unstable angina (UA), thrombosis, deep vein thrombosis, or pulmonary embolism. Heparin produces a dose-dependent prolongation of the clotting process measured by the activated partial thromboplastin time (aPTT). However, the anticoagulant effects of heparin are variable. Previous studies have reported wide subject variation in the dose of heparin required to achieve and maintain a therapeutic aPTT². A study, published in February 2009 in Circulation,³ further confirmed that only 33% of patients receiving heparin had therapeutic anticoagulation. The consequences of too high or too low a level of anticoagulation can be serious.⁴ In patients with acute ischemic syndromes, inadequate anticoagulation may lead to recurrent thrombosis, and significant bleeding has occurred in patients at supra-therapeutic doses of heparin. When a fixed dose of heparin is used as conjunctive therapy to thrombolysis or in the treatment of AMI, a substantial percentage of patients can be above or below the aPTT therapeutic range at any point in time.

Heparin is a naturally-occurring anticoagulant that when administered intravenously prevents the formation of clots and extension of existing clots within the blood. It is used for a number of different conditions. It is given as a continuous infusion for management of acute coronary syndromes, stroke, pulmonary emboli and venous thrombosis. Since the goal of therapy is to achieve a target range of anticoagulation rapidly and then maintain that level for a period of time, continuous infusions are monitored periodically and the dose is adjusted. Heparin dosing can be complicated by a number of factors, including illness that it is being used to treat. Various factors, including disease state can affect heparin pharmacokinetics and pharmacodynamics. Thus monitoring and dose adjustment are required to optimize therapy primarily for anticoagulation for cardiovascular conditions, including acute coronary syndromes, myocardial infarction, atrial fibrillation, cardiopulmonary bypass surgery (CABG), percutaneous coronary intervention (PCI), deep vein thrombosis and pulmonary embolism.

In the administration of heparin, the objective is to achieve an activated partial thromboplastin time (aPTT) value that is 1.5× to 2× the patient's baseline aPTT. As a result of the difficulty to correctly titrate heparin to any given patient, on average only 30% to 40% of patients achieve the desired aPTT range +/−15 seconds of administration during the course of therapy.⁵

The worldwide market for unfractionated heparin is estimated at $400 million.⁶ The US market for unfractionated heparin is about $146 million, It is a generic drug with Baxter, APP and Hospira comprising 80% of the market.⁷ Sales of heparin have maintained a steady growth over the past few years. From June 2006 to June 2007, total US heparin sales units grew by 6%.⁸ With the recent Baxter heparin recall early in 2008, the market (unit sales) has declined slightly as a result of less supply available in the market; however with manufacturers such as APP increasing production capacity, heparin supply should recover within the year.

Heparin is associated with many medication errors as a result of its complex pharmacologic response and large inter-patient variability in response. According to the United States Pharmacopoeia (USP) MED-MARX⁹, during a five year period from 2003 to 2007, heparin medication errors totaled 17,000 out of more than 50,000 anticoagulation related medication errors.¹⁰ The majority of heparin errors occur during administration at the bedside (47.6%) followed by prescribing errors (14.1%), dispensing (13.9%) and transcribing and documenting (18.8%). A majority of these errors resulted from a failure to follow procedures and protocols.¹¹ These errors all result in significant economic costs to the health care system.

Close monitoring of patients on heparin is extremely important: too low a dose of heparin can lead to under anticoagulation while too high a dose can lead to serious bleeding. It is also important to bring patients into range as quickly as possible to avoid adverse outcomes.¹² In studies of patients with acute coronary syndromes treated with intravenous heparin, increasing aPTT values were associated with increased bleeding episodes.¹³ At various times throughout therapy, only 50% of patients had aPTT values in the therapeutic range.¹⁴

Lower than required dosing levels of heparin can lead to episodes of thromboembolic complications in patients with acute coronary syndromes (ACS) or deep vein thrombosis while higher than required levels of heparin can lead to bleeding complications.¹⁵ In the recent “Can Rapid Risk Stratification of Unstable Angina Patients Suppress Adverse Outcomes with Early Implementation of the American College of Cardiology/American Heart Association Guideline (CRUSADE) initiative, it was observed that 49% of patients received excess dosing of unfractionated heparin leading to a significantly higher rate of major bleeding and need for transfusion as compared to patients who did not receive excess dosing.¹⁶

The problem has become so serious that the Joint Commission, which accredits all US hospitals issued a “Sentinel Event Alert”¹⁷ regarding the prevention of errors related to commonly used anticoagulants. Such alerts signal the need for immediate investigation and response for an event that carries a significant chance of a serious adverse outcome.

Current practices for the administration of heparin in an acute care setting involve many different steps and resources that can easily tax the hospital staff and lead to human error. General heparin dosing protocols (nomograms) may include the following steps: a standard initial bolus of heparin with a calculated infusion rate normally based on the patient's weight; instructions for drawing blood samples for partial thromboplastin time (aPTT) testing and orders for dosing adjustments in response to measured aPTT and optionally other values. The nurse will take a blood sample and send it to the central lab for analysis. The lab will provide the result to the nurse and the nurse will then evaluate the result and make the necessary adjustments to the dose based on the results. The nurse will check with the physician to verify dosing. Upon receiving approval from the physician, the nurse will make the necessary adjustment to the infusion rate. This process requires at least 1-2 hours to complete each time and is repeated every 4 to 6 hours over the course of approximately 2.5 days while the patient is receiving heparin.

As medication errors have continued to occur with heparin, sometimes causing serious complications, many hospitals and organizations have devised ways to try to minimize medication errors. Besides instituting nomograms for heparin administration, hospitals have tried other systems such as bar coding software that can identify and verify the drug and its concentration; inpatient anticoagulation services for heparin in which pharmacists run the services that provide daily pharmacy input on dosing and monitoring for patients on heparin; and automated medication dispensing systems.

The introduction of “smart” infusion pumps in the past few years have tried to address the issue of dosing errors before the patient suffers any negative effects. These smart pumps, which are still only used in approximately 50% of all hospitals in the US¹⁸, contain comprehensive drug libraries and standardized dosing units based on the specific acute care area of use. They also have dose calculators and alert systems if dosing falls out of pre-determined parameters or “guard-rails”. Nevertheless, recent reviews have concluded that many users of smart pumps bypass the safety features of the devices, and as a result medication errors continue to occur.¹⁹

Smart pumps attempt to prevent the nurse from inadvertently typing in a dose outside the standard dosing range. There is no provision for individualizing the dose for each patient, nor is there the ability to use a measure of patient response to adjust dosing. For medications with variable patient response (e.g. unfractionated heparin, insulin) the use of more individualized dosing and individualized adjustment according to a blood test has the potential to advance therapy and improve response.

Hospitals are increasingly concerned about medication errors. They are also in search of tighter control of critical parameters in the ICU, including anticoagulation and blood glucose. As a result, there is significant opportunity for a smart-controller that can integrate critical diagnostic assays and information to adjust patient dosing safely. With renewed focus on eliminating human error in drug administration of potent intravenous agents in the hospital, there is a large unmet need.

While previous systems have been described, see, e.g., Hillman et al., “Feedback Controlled Drug Delivery System”, U.S. Pat. No. 5,697,899, issued Dec. 16, 1997, Valcke et al., “Method and Apparatus For Closed Loop Drug Delivery”, U.S. Pat. No. 5,733,259, issued Mar. 31, 1998 and Gauthier et al., “Feedback Controlled Drug Delivery System”, U.S. Pat. No. 6,017,318, issued Jan. 25, 2000, they do not contain or integrate all of the advanced features in the current invention that are designed to further minimize medication errors and further improve the level of control.

### SUMMARY OF THE INVENTION

An integrated patient monitoring and control system is provided which includes a sampling infusion tubing set (SITS), the SITS being adapted for coupling to the patient to obtain a specimen from the patient, a sensor, the sensor being adapted to receive the specimen from the SITS and to analyze the sample, a medication control unit, the medication control unit receiving information from the sensor, and utilizing that information to determine medication dosing information specific to the patient, and a medication administration system, the medication administration system receiving the dosing information from the medication control unit, and adapted to cause administration of the medication to the patient. In one embodiment, the SITS is adapted for blood draw from the patient. Advantageously, the blood draw is performed in conjunction with a pneumatic pressure cuff, inflated so as to aid in blood draw.

In yet another embodiment, an automated blood sampling system, comprises a tourniquet, an indwelling catheter, a pressure measuring system, a pump, a disposable set, an optical source and detector, and a computer controlled adaptive algorithm. The system mechanizes blood draw by optimizing blood draw parameters such as by varying vacuum on the syringe plunger, slight manipulation, such as placement of the needle in the vein, all under control of the algorithm.

In another embodiment, a multi-parameter integrated patient monitoring and control system includes a sampling infusion tubing set (SITS), this set being adapted for coupling to the patient to obtain a specimen from the patient, a sensor, the sensor being adapted to receive the specimen from the SITS and to analyze the sample, the sensor including a first assay and at least a second assay, the assays testing for different medical conditions or different drugs, a medication control unit, the medication control unit receiving information from the sensor including information on the first and second assay, and utilizing that information to determine medication dosing information for the patient, and a medication administration system, the medication administration system receiving the dosing information from the medication control unit, the system including a first drug to be administered corresponding to the first assay and a second drug to be administered corresponding to the second assay, and adapted to cause administration of the medication to the patient. By way of example, the first assay could relate to blood clotting, e.g., aPTT, ACT, or Factor Xa value, and the first drug be heparin, and the second assay could relate to blood glucose level, and the second drug be insulin.

In yet another embodiment, a multi-parameter integrated patient monitoring and control system includes a SITS, the SITS being adapted for coupling to the patient to obtain a specimen from the patient, a sensor, the sensor being adapted to receive the specimen from the SITS and to analyze the sample, a medication control unit, the medication control unit receiving information from the sensor and at least one other patient information parameter, and utilizing that information to determine medication dosing information for the patient, and a medication administration system, the medication administration system receiving the dosing information from the medication control unit, and adapted to cause administration of the medication to the patient. In addition to the results of the first assay (that contains information relating to the patient response to the first drug being administered), a second item of patient information may be information from at least a second sensor or sensors or information relating to a first drug being administered, such as the drug level of the patient or information relating to the pharmacodynamic response of the patient to the first drug. The other patient information may also be the patient's vital signs, such as the blood pressure or heart rate of the patient, temperature and/or respiration rates.

### BRIEF DESCRIPTION OF THE DRAWINGS

FIG. 1 shows the cycle of the sample withdrawal set, the sensor, the medication control unit and the drug delivery technology.

FIG. 2 is a schematic block diagram of the main components of a heparin control algorithm.

FIG. 3 is a detailed block diagram of the system.

FIG. 4 is a flowchart showing overall operation of the system.

FIG. 5 shows a perspective view of the integrated patient management and control system for medication delivery.

FIG. 6 shows a perspective view of an alternate embodiment of the integrated patient management and control system for medication delivery.

FIG. 7A shows a top down view of an assay showing alternating assay regions. FIG. 7B shows a top down view of an assay showing four differing assays.

FIG. 8 shows a front view of a representative display system.

FIG. 9 is a flowchart of the single cuff implementation of the system and methods.

FIG. 10 is a flowchart of the multi-cuff implementation of the system and methods.

### DETAILED DESCRIPTION

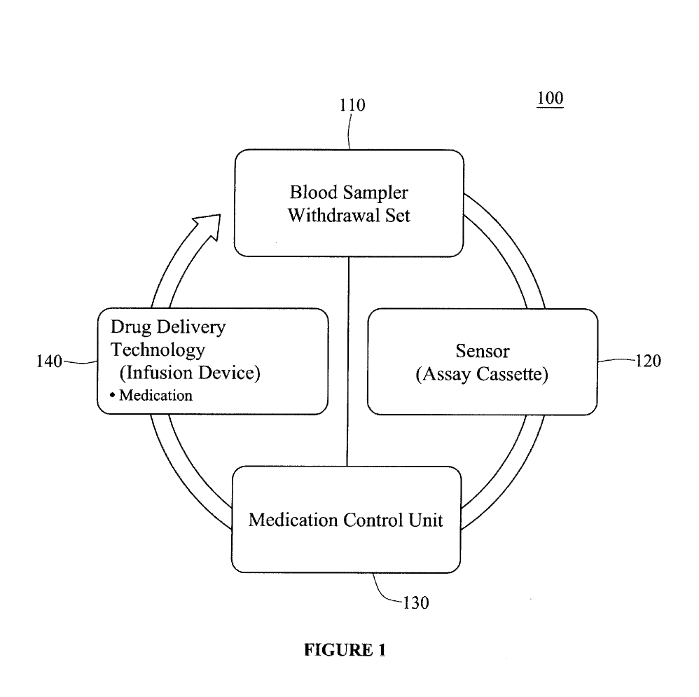

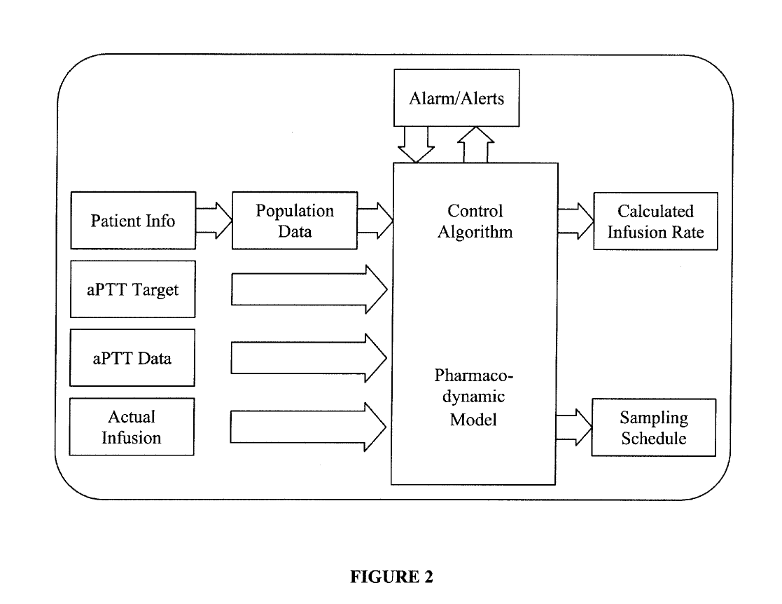

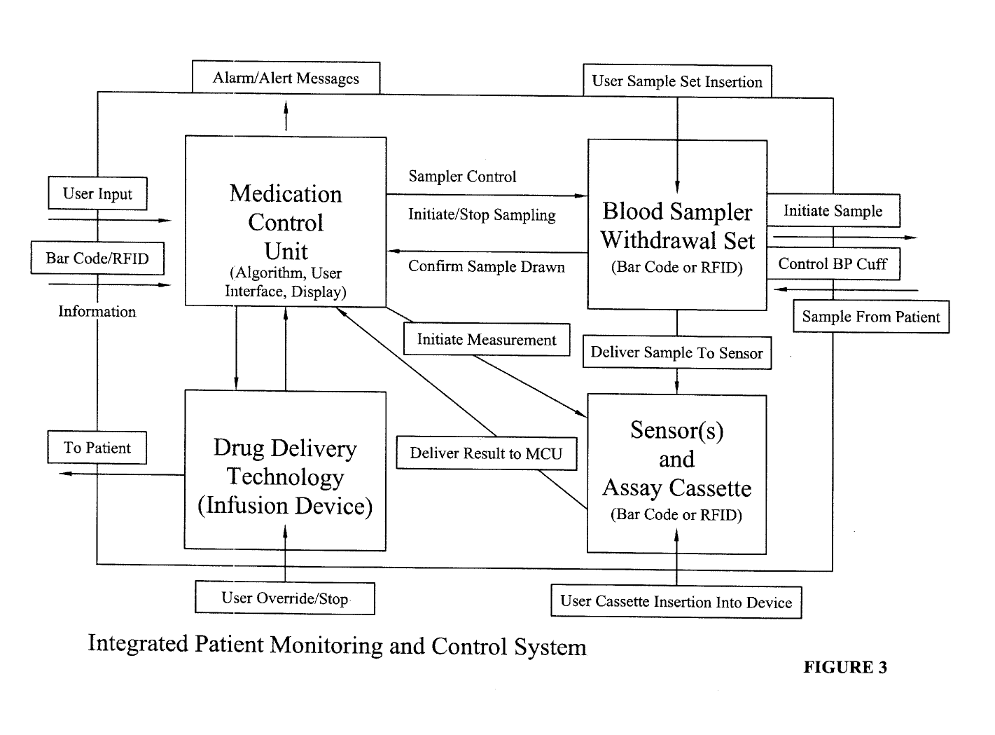

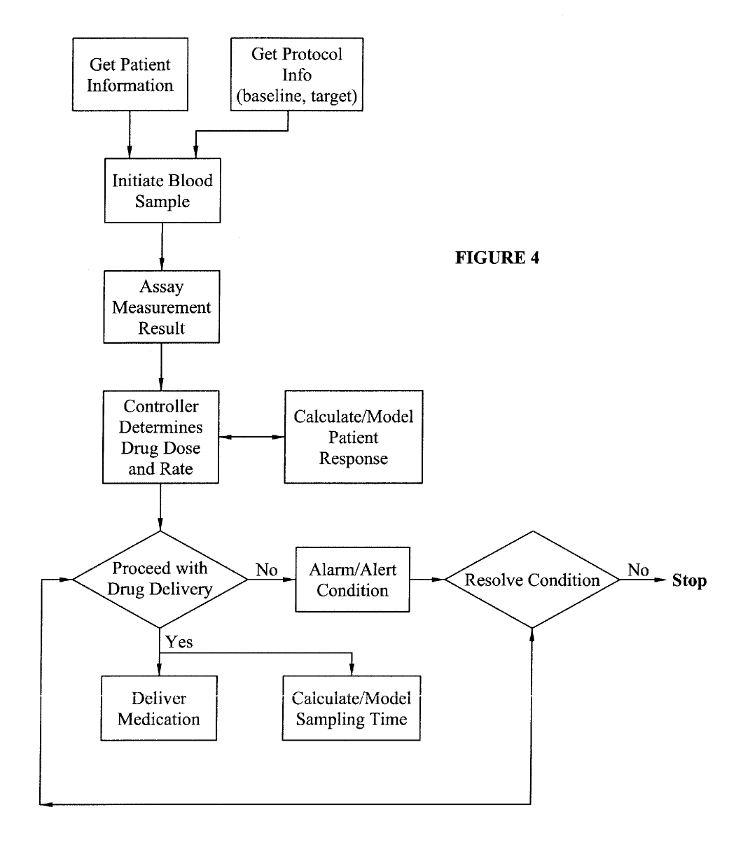

With particular reference to FIGS. 1, 2, 3, 4, 9 and 10, this invention describes an integrated patient measurement and control system 100 (IPMC) for delivering medications. The preferred elements of the system as depicted are the blood sampler/withdrawal tubing set (or SITS)-110, one or more sensors, 120 a medication control unit 130 and an integrated drug delivery technology 140 through which medication can be delivered.

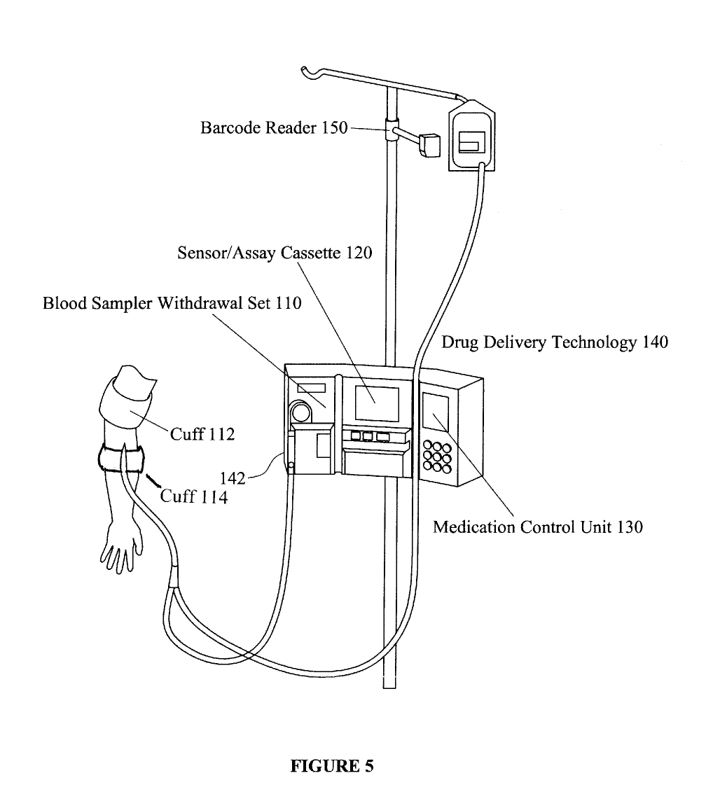

In one aspect, one of the key features of the IPMC System is an Integrated Drug Delivery Technology, shown in FIG. 5 is an integrated intravenous (IV) infusion pump. This integration minimizes the chance for communication errors that could occur with an external infusion device leading to potentially serious consequences such as infusion without proper feedback. Additional elements of the system include an integrated bar code reader (or RFID reader) 150 to read the name, dosage, and concentration of the medication to be delivered and patient ID to further minimize any medication delivery errors; intermittent sampling and control, and an inflatable tourniquet/constriction cuff that can be used in conjunction with the sampler device and medication control unit. The term cuff encompasses cuffs, including pneumatic cuffs, tourniquets or other forms of constriction devices. The system is capable of controlling different medications via interchangeable sensor and algorithms, or multiple medications through a multiplexed assay cassette.

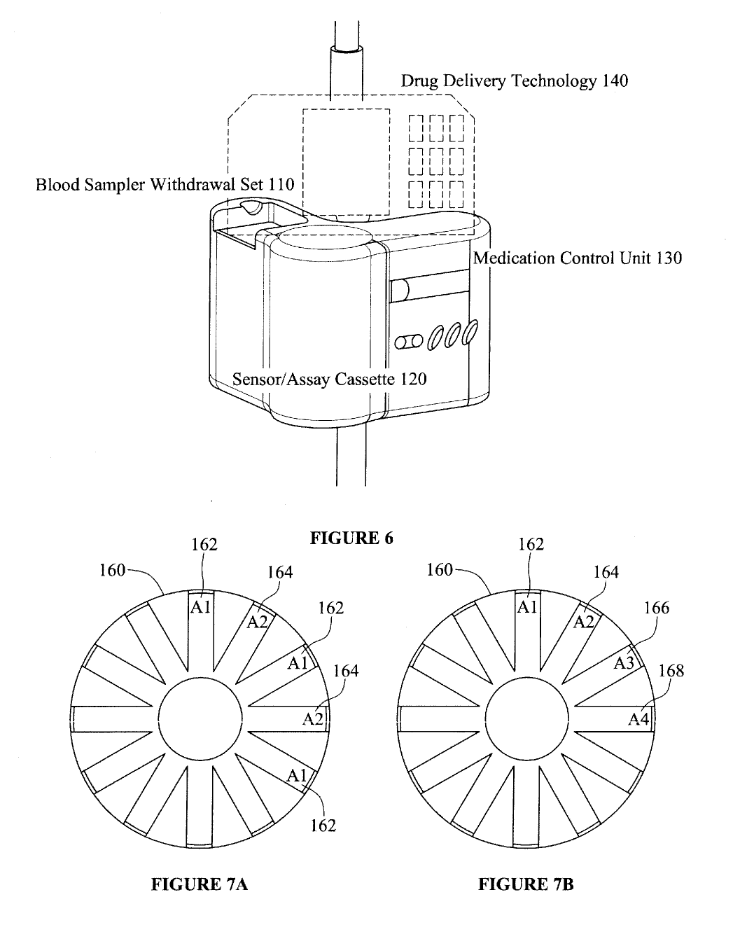

An alternative embodiment of the system is shown in FIG. 6, again containing integration of all of the elements described.

### Sampling System/Withdrawal Set.

The sampling system can be arranged to withdraw any biological fluid including blood, urine, interstitial fluid, or saliva. The preferred sample is blood. The sampling system preferably contains a bar code/RFID tag and interlock with the system to ensure patient safety and notify the medication control unit if any errors occur (e.g. occlusion, attempted removal, etc). The sampling system is capable of either intermittent sampling or could be adapted to continuous sampling based on the sensor(s).

The preferred embodiment of the sampling system incorporates an inflatable cuff 112 (blood pressure like cuff) and works in conjunction with the controller and sampler to ensure smooth withdrawal of blood. In one embodiment, two or more cuffs may be utilized. In the preferred embodiment of a multi-cuff system, one cuff 112 is located proximal of the point of insertion and the other cuff 114 is located distal to the point of insertion. The sampling system is coupled with a specific algorithm to inflate automatically prior to sampling (an automated corresponding to a tourniquet manually used for a lab blood draw) and use a sensing algorithm to set the pressure just above the systolic pressure to ensure a smooth draw and more frequent success to prevent vein collapse (especially in elderly).

The sampling system is preferably housed in a cassette that will fit into the device. In one aspect of the invention, an interlock system and optionally a bar code or RFID tag pair it with the IPMC.

In one system embodiment, the automated blood sampling system preferably comprises a tourniquet, an indwelling catheter, a pressure measuring system, a pump, a disposable set, an optical source and detector and a computer controlled adaptive algorithm. The tourniquet may be of any appropriate type, including hydraulic, pneumatic or mechanical, or any other fashion by which circumferential pressure can be applied to a limb. In one embodiment, the tourniquet optionally has a very low compliance, that is, it is relatively rigid system. Such a system has a relatively quick response time, with a fast on/fast off.

The tourniquet can be either above or below the point of insertion of the pressure monitoring catheter or system. If it is below the point of insertion, increased pressure may be utilized. The catheter may be “a single lumen catheter” or “a multi lumen catheter”. The pressure measuring system can be both invasive (via the indwelling catheter) or non-invasive (external pressure sensor). The catheter may be used to have a direct measure of venous pressure.

The pump may be of any type consistent with the application, such as a peristaltic pump, linear, rotary or cassette pump. A re-usable or disposable in-line transducer may be used to provide the pressure signal. If utilized, the disposable set interfaces with the pressure measuring system to provide real time or historic pressure measurement. Optionally, the pressure measuring system reads through the disposable set. In a preferred embodiment, pressure is measured transmurally, such as through use of an elastic segment of tubing laid across a strain gauge.

The optical sensor provides information to the adaptive algorithm. In the system, the presence of whole blood is indicated by absorbance of the optical signal, thus preventing it from reaching the optical detector. Optionally, the optical detector reads through the disposable set.

### The Multiple Tourniquet Embodiment

In one embodiment, multiple tourniquets are utilized adjacent the catheter. In the most preferred embodiment of this system, one tourniquet is disposed below the catheter and another is disposed above the catheter. Such a system provides the ability to meter the vessel dilation by adjusting each tourniquet pressure separately. While not limited to the following, various options for the pressure of the multiple cuffs are as follows:

in a first embodiment, applying pressure to cuff proximal to catheter,

in a second embodiment, applying pressure to cuff distal to catheter, keeping the pressure below the diastolic pressure,

in a third embodiment, for a distal location, use a pressure above diastolic pressure, or for a proximal approach use a pressure above systolic pressure.

in a fourth embodiment, alternate between both cuffs, which can be used to induce venous distension and dilation.

By limiting pressure to just below diastolic (or just above or both) safety is increased as arterial flow is still permitted. The enhanced safety aspect of a tourniquet that operates near or below diastolic offers significant safety advantage (no pain, hemostasis, etc) and if operated in a narrow pressure band, the time to reach and/or adjust tour pressure is quite short, which is an advantage to ‘manipulate’ the vessel diameter somewhat.

As pressure in the vein drops, the pump rate (and therefore its vacuum) also drops to prevent vein collapse. As the pressure cuff enhances venous pressure, the pump speeds up. A goal is to maintain constant local venous pressure in the area of the catheter tip, most particularly proximal to the nearest valve in the vein. As venous pressure rises, so does the withdrawal rate of the pump. It may exceed baseline pressure (venous pressure with no external fluid moving in or out of the catheter) depending on the effect of tourniquet. Optionally, ramp rates may be varied.

This mechanically moves the catheter tip away from whatever might be blocking it by using reactionary force. If the infusion is fast, the catheter tip will have a force on it that moves it away from the valve or venous wall. Again, this might be in conjunction with the tourniquet manipulations.

### Systems and Methods for Enhancing Vein Lumen Diameter

In yet another embodiment, the algorithm alerts an infusion pump, fluidically connected to the indwelling catheter, to infuse saline or other fluid at a high rate. One effect of fast infusion is to enhance vein lumen diameter.

First, an infusion of saline may be used to enhance venous diameter. Optionally, this infusion may be used in conjunction with some tourniquet pressure.

Second, a local vasodilator may be used rather than saline if it does not interfere with the aPTT infusion, and is effective at dilating a vein. While saline may result in physical distention, other infusates have a dilating effect, e.g., nitroprusside, or other vasodialator known to those skilled in the art. Enhancers of nitrous oxide, delivered locally, may provide a vasodilatation effect. A very low concentration may be utilized. A fluid that produces very local vasodilation may be used to enhance sample withdrawal success rate.

Third, in one embodiment, the pressure to the tourniquet is oscillated. The oscillations may be rapid or slow. One advantageous result of the oscillations is to enhance venous dilation.

Fourth, a special multi-orifice catheter may be employed to avoided positional effects of the catheter opening.

### Infusion Systems and Methods

The algorithm may alert an infusion pump, fluidically connected to the indwelling catheter, to infuse saline or other fluid at a high rate to displace the catheter tip from the venous wall to enhance sample withdrawal.

Such an infusion results in mechanical movement of the catheter tip away from whatever might be blocking it by using a reactive force. Upon fast infusion the catheter tip will have a force on it that moves the catheter away from the valve, venous wall or other obstruction. Optionally, this technique may be used in conjunction with the tourniquet manipulations.

### Feedback Sensor(s)

The IPMC 110 is a modular system with the capability of providing feedback on different parameters from different medications or on more than one parameter (e.g., drug level, pharmacodynamic response) simultaneously. This is achieved by having the sensor be interchangeable in the device or by a sensor that can be used with more than one assay parameter. One embodiment, shown below in FIGS. 7A and 7B, is a cassette 160 which consists of multiple assays for different assays (e.g., a1 162, a2 164 (alternating); or a1 162, a2 164, a3 166, a4 168 (in sequence)). Thereby multiple assay parameters (e.g. aPTT, glucose concentration, potassium level) can be detected in sequence. The embodiment below preferably interlocks with the system and contains a barcode/RFID tag to ensure that the correct parameters are being measured.

In another aspect of the invention of the system, vital signs monitoring (e.g. ECG, blood pressure, Sp02) is integrated into the overall monitoring of the safety and state of patient. The blood pressure and heart rate can be analyzed using the cuff 112 that is part of the sampling system.

### Algorithm and Medication Control Unit (MCU)

The IPMC System is based on intermittent sampling or if the sensor allows, continuous measurement. It is important to note that the sampling system may take intermittent samples, and the MCU 130 uses algorithms to reconstruct patients state, response and then calculate drug delivery rate based on intermittent samples. In addition, the optimal sampling time to take a sample can be determined by analyzing the response of the patient and if patient response is unexpected (e.g., in wrong direction) the medical delivery is halted and an alert or alarm is raised.

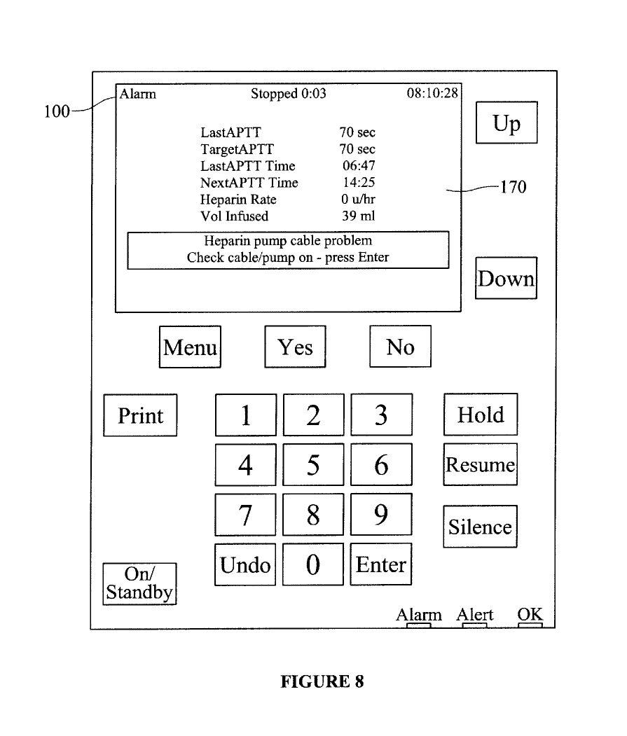

There is also an alarm/alert infrastructure/supervisory system 100 to oversee the entire MCU. If all aspects of the IPMC System are functioning there is a “green light” and delivery proceed. If there is an alert, (e.g., a non-critical problem that is potentially correctable) has been detected (e.g. sampling error, communication error, etc.) a yellow alert and audible alarm occurs. If a serious condition occurs (incorrect infusion rate, multiple missed samples, disconnected line) then the system immediately goes into alarm (red light, audible alarm, communication to central station). FIG. 8 shows a representative display of a monitor 170 for the system.

The adaptive algorithm controls the pneumatic or mechanical tourniquet to apply pressure or release pressure to the subject's extremity proximal (closest to the heart) to the indwelling catheter. In one implementation, the adaptive algorithm controls the tourniquet pressure based on real time and historic data both within patient and based on population data. The adaptive algorithm preferably adjusts the withdrawal rate of the pump based on real time and historical measurement provided by the pressure measuring system.

A heuristic algorithm is optionally included that ‘learns as it goes’ on a per-subject basis. Such a system preferably starts with a population basis.

Real-time venous pressure measurements may be included in the algorithm, if available. Alternatively, pressure may be measured indirectly, such as via external strain gauge.

The algorithm attempts to optimize the sample integrity, such as by maximizing the sample draw speed, to minimize sample time in the sample tube, to avoid sample degradation, e.g., degradation of aPTT measurements.

In yet another embodiment, the adaptive algorithm controls both the tourniquet pressure and the with-drawl rate based on real time and historic pressure data. The combination of these two ideally results in better sample draw than either factor individually. The adaptive algorithm may compensate for inferred venous pressure drop by altering the withdrawal rate. As pressure in the vein drops, the pump rate (and therefore its vacuum) also drops to prevent vein collapse. As the pressure cuff enhances venous pressure, the pump speeds up. The goal is to maintain constant local venous pressure in the area of the catheter tip and certainly proximal to the nearest valve in the vein. As venous pressure rises, so does the withdrawal rate of the pump, indeed, it may exceed baseline pressure (venous pressure with no external fluid moving in or out of the catheter) depending on the effect of tourniquet. Other variations may be utilized, such as ramp rates.

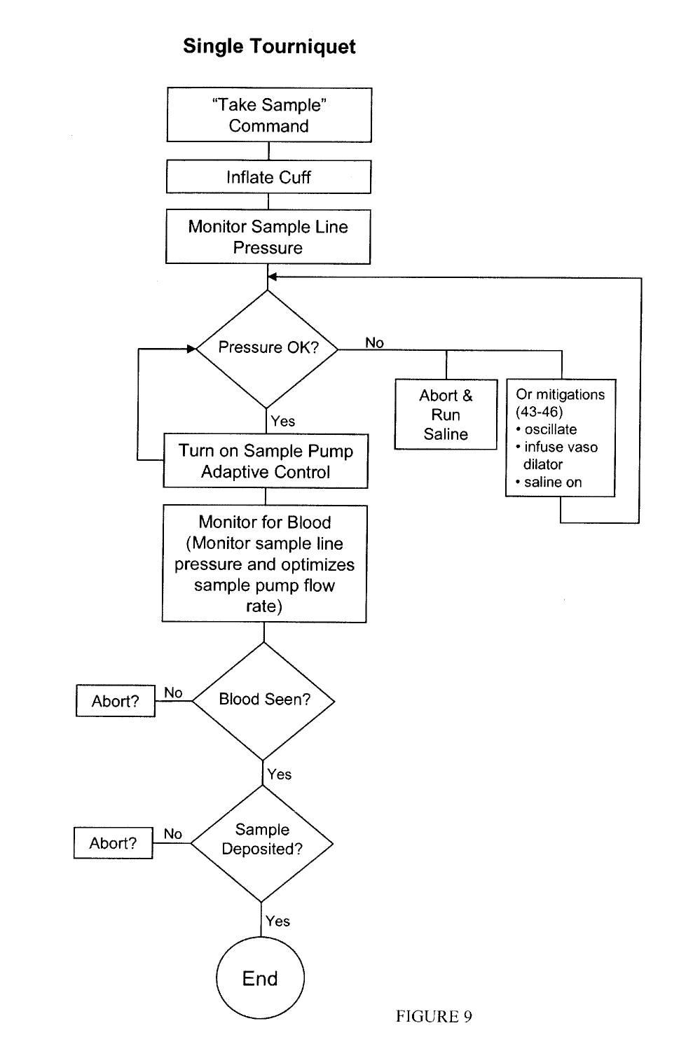

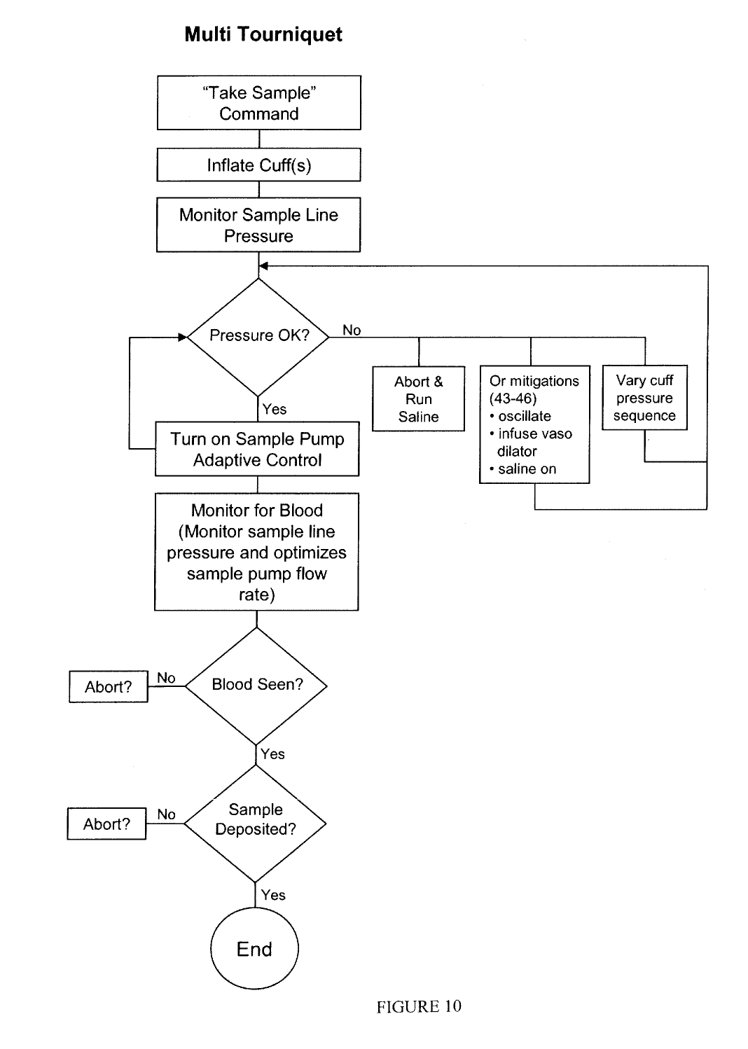

The adaptive algorithm may be implemented on a microprocessor or microcontroller. FIGS. 9 and 10 show flow charts for possible implementations of the systems and methods of the inventions. In FIG. 9, the system initially issues a “Take Sample” command. Next, the cuff is inflated. In the third step, the sample line pressure is monitored. If the pressure is within acceptable limits, the system proceeds to turn on the sample pump under adaptive control. At least while the pump is on, the system monitors for blood in the line. Preferably, the sample line pressure is also monitored, which is then used to optimize the sample pump flow rate. If no blood is seen, the sample is then deposited, and the system can then end. If blood is seen, an abort is an option. If (after step 3, above) the pressure is not within acceptable limits, the system any either (1) abort and run saline in the line, or (2) attempt various mitigation routines as discussed herein, including but not limited to oscillation of the pressure, infusion of a vaso dilator, or to turn the saline on.

The process of the multi-tourniquet system is as described for FIG. 9, but further includes the option after the third step in the event the pressure is not within acceptable limits, to vary the cuff pressure sequence. Possible sequences could include, but are not limited to, inflate the proximal cuff, recheck the pressure, and if it is not within acceptable limits, to inflate the distal cuff, and deflate the proximal cuff. If the pressure is still not within acceptable limits, the distal cuff could be deflated and the procedure repeated. These sequences may be performed in any order or combination or permutation.

### System and Method Control

In one embodiment, the tourniquet pressure is limited to approximately or slightly lower than diastolic pressure to prevent hemostasis in the extremity.

By limiting pressure to just below diastolic (or just above or both) we are increasing safety as arterial flow is still permitted. The enhanced safety aspect of a tourniquet that operates near or below diastolic offers significant safety advantage (no pain, hemostasis, etc) and if we operate in a narrow pressure band the time to reach and/or adjust tour pressure may be quite short. This can advantageously serve to ‘manipulate’ the vessel diameter.

### Medication Delivery Technology

The medication delivery technology optionally consists of intravenous infusion pumps 142, syringe pumps, implantable pumps, transdermal iontophoretic systems. The preferred embodiment is an intravenous infusion pump. The preferred delivery route is intravenous, but other portals such as intrarterial, transdermal, peritoneal, subcutaneous, or buccal could also be used.

In the preferred embodiment, the pump is an integral part of the system rather than connected by an interface. This prevents any potential safety issues including 1) communication errors between devices, 2) incorrect information being sent between devices, 3) loss of control of device, 4) undetected error that is missed by pump and not detected by the medication control unit. Optionally, the system, will contain a bar code reader 150 that can read the identity of the medication being delivered as well as its concentration, and patient for whom it is intended.

### Alerts and Alarms

Optionally, a safety algorithm alerts the caregiver that a sample can not be obtained unless a set of predefined conditions are met. Various alerts and alarms may be used. A clinical alert can also be incorporated to notify a clinician that drug is scheduled to be delivered, and require approval by the physician (directly or through a remote connection) before administration.

### Applications

The systems and methods described herein may be used for automated blood sampling, and then used in combination with other systems, methods and applications. Of particular utility are closed loop systems which use the described automated blood sampling in combination with a diagnostic assay to provide an analysis of the blood, and where that analysis is used in providing a drug or other material to the patient. Most preferably, the closed loop system is fully automated from the blood sampling, to the diagnostic assay, to the provision of drug delivery.

### Additional Aspects

The system preferably includes telemetry (either wired via ethernet or like, or wireless like bluetooth or WIFI) to communicate information to central station. The system has the ability to pair the system with the patients instructions to make sure the right patient is being started on the right drug.

While various embodiments have been described herein, they may be used in combination with multiple embodiments. The embodiments may be combined in order to optimize successful sampling and control.

Although the foregoing invention has been described in some detail by way of illustration and example for purposes of clarity and understanding, it will be readily apparent to those of ordinary skill in the art in light of the teachings of this invention that certain changes and modifications may be made thereto without departing from the spirit or scope of the appended claims.

### REFERENCES

¹The Joint Commission Sentinel Event Alert: Preventing errors relating to commonly used anticoagulants Issue 41, Sep. 24, 2008.
²Granger C B, Hirsh J, Califf R M et al. for the GUSTO-I Investigators. Activated partial thromboplastin time and outcome after thrombolytic therapy for acute myocardial infarction: results from the GUSTO-I Trial. Circulation. 1996; 93:870-878.
³Cheng S, Morrow D A, Sloan S, Antman E M, Sabatine M S. Predictors of initial nontherapeutic anticoagulation with unfractionated heparin in ST-segment elevation myocardial infarction. Circulation. 2009 Mar. 10; 119(9):1195-202. Epub 2009 Feb 23.
⁴Anand et al. Relationship of Activated Partial Thromboplastin Time to Coronary Events and Bleeding in Patients with Acute Coronary Syndrome Who Receive Heparin. Circulation. 2003; 107:2884-2888.
⁵Cannon et al. Automated Heparin Delivery System to Control Activated Partial Thromboplastin Time. Circulation. 1999; 99:751-756.
⁶Alchemia's generic fondaparinux a potential beneficiary of heparin product recall. Alchemia Ltd. press release: Mar. 27, 2008. <http://www.alchemia.com>
⁷IMS National Sales Perspective Report. IMS Health Inc. June 2008.
⁸ Ibid.
⁹MEDMARX® is a national database that tracks and trends adverse drug reactions and medication errors.
¹⁰C. Peterson, C. Ham, T. Vanderveen. Improving Heparin Safety: A Multidisciplinary Invited Conference. Hospital Pharmacy, Vol. 43, No. 6, pp 491-497.
¹¹Ibid.
¹²Granger C B, Hirsh J, Califf R M et al. for the GUSTO-I Investigators. Activated partial thromboplastin time and outcome after thrombolytic therapy for acute myocardial infarction: results from the GUSTO-I Trial. Circulation. 1996; 93:870-878.
¹³Anand et al. Relationship of Activated Partial Thromboplastin Time to Coronary Events and Bleeding in Patients with Acute Coronary Syndrome Who Receive Heparin. Circulation. 2003; 107:2884-2888.
¹⁴Ibid.
¹⁵T. K. Gandhi et al. Protocols for High-Risk Drugs: Reducing Adverse Drug Events Related to Anticoagulants. Agency for Healthcare Research and Quality (AHRQ).
¹⁶T Y Wang, E Peterson, M Ohman et al. Excess Heparin Dosing Among Fibrinolytic-treated Patients with ST-Segment Elevation Myocardial Infarction. American Journal of Medicine (2008) 121:805-810.
¹⁷The Joint Commission Sentinel Event Alert: Preventing errors relating to commonly used anticoagulants Issue 41, Sep. 24, 2008.
¹⁸C. Peterson, C. Ham, T. Vanderveen. Improving Heparin Safety: A Multidisciplinary Invited Conference. Hospital Pharmacy, Vol. 43, No. 6, pp 491-497.
¹⁹Smart Pumps Are Not Smart On Their Own. Institute for Safe Medication Practices Newsletter, Apr. 19, 2007.

What is claimed:

1. An automated blood sampling system, comprising:
a tourniquet,
an indwelling catheter,
a pressure measuring system,
a pump,
a disposable set,
an optical source and detector, and
a computer controlled adaptive algorithm.

2. The automated blood sampling system of claim 1 wherein the tourniquet is a pneumatic.

3. The automated blood sampling system of claim 1 wherein the tourniquet is hydraulic.

4. The automated blood sampling system of claim 1 wherein the tourniquet is mechanical.

5. The automated blood sampling system of claim 1 wherein the tourniquet has low compliance.

6. The automated blood sampling system of claim 1 wherein the pump is a peristaltic pump.

7. The automated blood sampling system of claim 1 wherein the catheter is a single lumen catheter.

8. The automated blood sampling system of claim 1 wherein the catheter is a multi lumen catheter.

9. The automated blood sampling system of claim 1 wherein the pressure measuring system is invasive.

10. The automated blood sampling system of claim 9 wherein the invasive pressure measuring system includes an indwelling catheter.

11. The automated blood sampling system of claim 1 wherein the pressure measuring system is non-invasive.

12. The automated blood sampling system of claim 11 wherein the non-invasive pressure measuring system includes an external pressure sensor.

13. The automated blood sampling system of claim 1 wherein the adaptive algorithm adjusts the withdrawal rate of the pump based on real time and historical measurement provided by the pressure measuring system.

14. The automated blood sampling system of claim 1 wherein the disposable set interfaces with the pressure measuring system to provide real time or historic pressure measurement.

15. The automated blood sampling system of claim 1 wherein the optical sensor provides information to the adaptive algorithm the presence of whole blood by absorbance of the optical signal thus preventing it from reaching the optical detector.

16. The automated blood sampling system of claim 1 wherein the optical detector interfaces with the disposable set.

17. The automated blood sampling system of claim 1 wherein the pressure measuring system interfaces with the disposable set.

18. The automated blood sampling system of claim 1 wherein the adaptive algorithm controls the tourniquet pressure based on real time and historic data both from the specific patient and based on population data.

19. The automated blood sampling system of claim 1 wherein the pressure to the tourniquet is oscillated to enhance venous dilation.

20. The automated blood sampling system of claim 19 wherein the oscillation is slow.

21. The automated blood sampling system of claim 19 wherein the oscillation is fast.

22. The automated blood sampling system of claim 1 wherein the tourniquet pressure is limited to approximately or slightly lower than diastolic pressure.

23. The automated blood sampling system of claim 1 wherein the adaptive algorithm controls both the tourniquet pressure and the withdrawal rate based on real time and historic pressure data.

24. The automated blood sampling system of claim 1 wherein the adaptive algorithm compensates for inferred venous pressure drop by altering the withdrawal rate.

25. The automated blood sampling system of claim 1 wherein a safety algorithm alerts the caregiver that a sample can not be obtained unless a set of predefined conditions are met.

26. The automated blood sampling system of claim 1 wherein the algorithm alerts an infusion pump, fluidically connected to the indwelling catheter, to infuse saline or other fluid at a high rate to enhance vein lumen diameter.

27. The automated blood sampling system of claim 1 wherein the algorithm alerts an infusion pump, fluidically connected to the indwelling catheter, to infuse saline of other fluid at high rate to displace the catheter tip from the venous wall to enhance sample withdrawal.

28. The automated blood sampling system of claim 1 wherein multiple attributes work in to optimize sampling success.

29. The automated blood sampling system of claim 1 further including a second tourniquet adapted to be placed distal to the intended insertion site of the catheter.

30. The automated blood sampling system of claim 1 wherein the algorithm controls monitoring and control functions.
